(19) United States
(12) Patent Application Publication
(10) Pub. No.: US 2025/0186195 A1
(43) Pub. Date: Jun. 12, 2025

(54) POLYMER VALVE LEAFLET MATERIAL, PREPARATION METHOD FOR INTEGRALLY MOLDING SAME, AND USE THEREOF

(71) Applicant: VENUS MEDTECH (HANGZHOU) INC., Hangzhou (CN)

(72) Inventors: Renzheng Ma, Hangzhou (CN); Lichao Wang, Hangzhou (CN); Yuting Zhu, Hangzhou (CN); Dajun Kuang, Hangzhou (CN); Xing Wei, Hangzhou (CN)

(21) Appl. No.: 19/059,329

(22) Filed: Feb. 21, 2025

Related U.S. Application Data
(63) Continuation of application No. PCT/CN2023/106864, filed on Jul. 12, 2023.

(30) Foreign Application Priority Data
Aug. 22, 2022 (CN) 202211008231.8

(51) Int. Cl. A61F 2/24; D01D 5/00; D01F 6/70

(52) U.S. Cl. CPC A61F 2/2415; A61F 2/2418; D01D 5/0069; D01D 5/0076; D01F 6/70; D10B 2331/10; D10B 2509/06

(57) ABSTRACT

Disclosed are a polymer valve leaflet material, a preparation method for integrally molding the same, and use thereof. The preparation method includes: a spinning step, electrospinning a first polyurethane solution to a receiving device to obtain a prefabricated film on the receiving device; and a compositing step, compositing second polyurethane with the prefabricated film to obtain the polymer valve leaflet material. The present disclosure solves the problems of uneven thickness of various parts of the leaflets, weak points inside the leaflets, and easy tearing of the leaflets at weak points in the existing impregnation process, thereby addressing the issues of easy creep, relatively low overall strength and poor tear resistance of the existing leaflets.

### CROSS-REFERENCE TO RELATED APPLICATIONS

The present application is a Continuation application of PCT Application No. PCT/CN2023/106864, filed on Jul. 12, 2023, which claims the priority of Chinese Patent Application No. 202211008231.8, filed on Aug. 22, 2022, the entire contents of which are hereby incorporated by reference.

### TECHNICAL FIELD

The present disclosure relates to the technical field of interventional materials, and in particular to a polymer valve leaflet material, a preparation method for integrally molding the same, and use thereof.

### BACKGROUND

Currently, heart valve prostheses used in clinical practice are classified into two categories: artificial mechanical valves and biological valves. Mechanical valves have a long service life and good stability, but they suffer from poor biocompatibility and require long-term anticoagulation treatment. Biological valves have good biocompatibility, but they are prone to calcification and degradation, which leads to poor valve durability, with a lifespan of only 10 to 15 years. In addition, the source of materials is limited, and their homogeneity is poor, requiring a lot of manpower for screening and thus resulting in high costs and low utilization rates.

In recent years there has been a rise of polymer valves, with leaflets made of polymer materials, which are easy to process and offer good uniformity of performance. The main polymer materials currently used for leaflets include polyurethane and polyetheretherketone, etc, which have been widely used in the development of implantable medical devices due to their excellent biocompatibility and suitability as valve leaflet materials. However, polymer materials are prone to creep, which can cause the leaflets to elongate and deform during use, thereby affecting the fluid dynamic performance of the valve. Moreover, polymer materials have poor edge tear strength and are prone to cracking. This is one of the main problems restricting the development of polymer valves.

In order to solve the existing problems, patent literature has disclosed that the mechanical properties of the valve leaflets have been improved by means of multilayer compositing and fiber reinforcement, thereby improving the durability of the valve. For example, as disclosed in Chinese patent publication No. CN 113813080 A, multiple layers of nano-fiber membranes are composited together to prepare valves with high elasticity, high mechanical strength and fatigue resistance. Additionally, nano-fibers promote the endothelialization process and exhibit anti-thrombotic. However, multi-layer composites suffer from poor interlayer bonding, and the valve is flat which relies on stress to form a 3D shape. As a result, the valve is subjected to higher stress during the cardiac cycle, which can easily lead to calcification. This may cause poor durability, delamination, and detachment of the valve.

### SUMMARY

The present disclosure provides a polymer valve leaflet material, a preparation method for integrally molding the same, and use thereof, which address issues of uneven thickness of various parts of the leaflets, weak points within the leaflets, and easy tearing of the leaflets at weak points in the existing impregnation process, thereby addressing the issues of easy creep, relatively low overall strength and poor tear resistance of the existing leaflets.

A method for preparing a polymer valve leaflet material by integral molding includes:
a spinning step, electrospinning a first polyurethane solution onto a receiving device to obtain a prefabricated film on the receiving device; and
a compositing step, compositing a second polyurethane with the prefabricated film to obtain the polymer valve leaflet material.

In the preparation method of the present disclosure, both the electrospun layer obtained on the surface of the receiving device and the leaflet obtained by drying after impregnation compositing are three-dimensional integral molded. This ensures that the leaflet is subjected to less stress during a cardiac cycle and slows down the occurrence of calcification. In addition, the impregnation process is performed on the rough electrospun layer, which inhibits the uneven thickness of the polyurethane caused by gravity, thereby avoiding the formation of weak points within the leaflet. Besides, the electrospun layer improves the creep resistance and edge tear resistance of the valve leaflet, thereby extending the service life of the valve.

In the solvent system of the compositing step of the second polyurethane and pre-polymer valve, the first polyurethane serves as the material of the scaffold, while the second polyurethane serves as the the material of the composite layer. The solubility requirements for the first polyurethane and the second polyurethane are different. As a general principle, it is necessary to ensure that the second polyurethane can be well dissolved in the solvent system of the compositing step, while the prefabricated film prepared from the first polyurethane still maintains the integrity of the scaffold.

Optionally, a hard segment content of the second polyurethane is 5% to 10% less than that of the first polyurethane.

Further optionally, the first polyurethane is a polyurethane with a hard segment content of 35% to 55%.

Still further optionally, the first polyurethane, in addition to having a hard segment content of 35% to 55%, is poorly soluble in solvent systems such as DMF, DMAc, and DMSO, which can be used in the compositing step.

Even further optionally, the first polyurethane is a polyurethane with a hard segment content of 40% to 55%, wherein the hard segment is partially composed of diamines.

Optionally, the second polyurethane is biocompatible; further, the second polyurethane is selected from PDMS-PU.

The first polyurethane and second polyurethane complying with the above requirements may be prepared by the publicly available preparation methods in the industry, or may be obtained through commercial purchase.

In the electrospinning step, the receiving device serves as a carrier for integrally molding the prefabricated film and is structured so as to facilitate the three-dimensional molding of the prefabricated film.

As a first optional scheme of the receiving device, the receiving device includes a head part with inclined walls, and the shape of each inclined wall is adapted to the shape of the corresponding leaflet in the transition state.

Optionally, the leaflet includes a fixed edge and a free edge, and in the transition state (also understood as a semi-open state), two adjacent leaflets are partially close to each other, and the close portions include:
the portion of the free edge adjacent to the fixed edge of each leaflet; and
the portion of the free edge of each leaflet adjacent to the center of its own length.

The fixed edges of the leaflets are used to sew the leaflets to the stent, and the free edges of each leaflet cooperate with each other to control the blood flow channel.

Taking the trileaflet valve as an example, when the leaflets are in the closed state, one side of the length center of the free edge of a leaflet is in contact with the free edge of one of the adjacent leaflets, and the other side of the length center is in contact with the free edge of another adjacent leaflet, as shown in FIG. 1; when the leaflets are in the open state, the free edges of the two adjacent leaflets are separated from each other to allow the blood flow passage, as shown in FIG. 2.

From the perspective of the deformation amplitude of the leaflets, the closed state and the open state can be understood as the limit states of leaflet deformation. The shape of the receiving device in the present disclosure corresponds to the transition state of the leaflets. After molding, the extreme stress in the deformation process can be reduced, which improves fatigue resistance to a certain extent, prolongs service life, and avoids tearing caused by premature attenuation of mechanical properties.

When the leaflets are in the closed state, the fitting length of the free edges between two adjacent leaflets is substantially half of the total length of the free edge. In the present disclosure, the total length of the two portions that are partially close to each other is approximately 0.1 to 0.4 times the total length of the free edges.

Further optionally, the shape of the inclined wall corresponds to the leaflet being in a state where the opening area is ¼ to ¾ of the full opening.

The valve leaflets involved in this disclosure include but are not limited to the pulmonary valve, mitral valve, tricuspid valve, and aortic valve according to their application scenarios.

Optionally, the receiving device further comprises a tail part connected with the head part, the tail part is a rotating body as a whole, and the outer peripheral surface of the tail part is provided for electrostatic spinning to form a skirt connected with the fixed edge of the leaflet. The skirt may be sewn on the inner side of the stent to further improve the effect of preventing peripheral leakage.

Optionally, the head and tail parts are formed in one piece.

Optionally, the end face of the head part is planar.

The receiving device of this scheme may be either stationary or rotating during operation, with a general rotation speed of 0.5 r/min to 20 r/min. An electrospun layer (or also including the skirt) with the same shape corresponding to the half-open state of the leaflet is formed on the outer wall of the receiving device through electrospinning, which serves as a prefabricated film and is then composited with the second polyurethane.

In this solution, it is preferred to maintain the shape of the prefabricated film during the compositing step, for example, by dip-coating it together with the receiving device to avoid unexpected deformation of the prefabricated film when compositing.

Alternatively, after being separated from the receiving device, the shape of the prefabricated film is maintained by a shape-matching support member. In order to ensure the compositing effect, hollow structures, such as a grid or micropores, may be used for the part where the supporting member contacts the prefabricated film, to facilitate the penetration of the second polyurethane.

In the second optional scheme of the receiving device, the outer circumference of the receiving device is a cylindrical surface, which rotates at high speed during operation. A cylindrical prefabricated film may be formed by electrospinning, for which the following subsequent operations may be performed.

For the cylindrical prefabricated film, one axial end thereof is bent inwardly before compositing, and the shape after bending corresponds to the closed state and/or transition state of the leaflets.

Since there may be certain internal stress in the bent portion, the valve after compositing may be heat-set to correspond to the closed and/or transitional state of the leaflets.

In both the first and second receiving device schemes, all leaflets and skirts may be formed into a one-piece structure, eliminating the need for splicing between the parts and allowing for better mechanical properties.

Optionally, in the second receiving device scheme, the receiving device has a rotation speed of 1500 to 2500 r/min. This rotation speed range is conducive to the formation of anisotropy of the leaflet material, for example, to form a structure with fiber orientation similar to a biological valve, which improves the tear resistance at the line-hole site after being sewn to the stent, and in the vicinity of the free edge, the fiber orientation is approximately the same as the length direction of the free edge, which can further enhance fatigue resistance.

During the electrospinning process, the first polyurethane is configured into a solution. Optionally, the mass percentage concentration of the first polyurethane in the first polyurethane solution is in a range of 8% to 18%. The solvent should be chosen to facilitate the full dissolution of the first polyurethane, such as a THF/DMF (N,N-dimethylformamide) mixed solvent with a volume ratio of 1:1 to 5:1. (THF: tetrahydrofuran; DMF: N,N-dimethylformamide).

As a more specific example, the first polyurethane is stirred in a THF/DMF mixed solvent with a volume ratio of 1:1 to 5:1 for 4 to 16 hours to prepare a homogeneous solution with a concentration range of 8 wt % to 18 wt %.

Optionally, during the electrospinning process, the control conditions are set as follows: a humidity of 45 to 55%, a voltage of 10 to 25 kV, a propulsion speed of 0.5 to 1 mL/h, with a spinning distance of 15 to 25 cm, and a needle gauge of 10 G to 30 G.

During the electrospinning process, when selecting the first receiving device scheme as described above, it is further optional to apply an electric field through an electrode directly above the head part of the receiving device (as shown in the orientation of the figure). The electrode includes a ring-shaped negative electrode and a positive electrode located at the center of the negative electrode, the plane where the negative electrode is located is perpendicular to the axis of the receiving device, and the positive electrode is aligned with the axis of the receiving device.

The distance between the electrode and the head part of the receiving device is preferably in a range of 1 to 3 cm along the height direction.

The inner diameter of the negative electrode is in a range of 10 to 30 mm, and the diameter of the positive electrode is in a range of 2 to 5 mm.

Further optionally, during the electrospinning process, the receiving device is located in a magnetic field environment, and the magnetic field lines are directed from the receiving device upward to the electrode.

During the electrospinning process, the spinning liquid nozzle is positioned above the electrode, that is, the electric field is located between the spinning liquid nozzle and the receiving device. After being ejected from the nozzle, the spinning liquid enters the electric field, which provides a radially divergent deflection force to the fiber filaments, changing their movement direction and cutting through the magnetic field lines, thus providing a circumferential twisting force to the fiber filaments under the action of the magnetic field. With the coordinated cooperation of the electric field and the magnetic field, the arrangement of the fiber filaments is more similar to the natural leaflet morphology, further improving the fluid dynamic performance of the leaflet, and the fluid results thereof are close to those of unreinforced pure polyurethane materials.

Optionally, the voltage between the positive and negative electrodes is in a range of 15 to 30 kV.

Optionally, the magnitude of the magnetic field is in a range of 0.01 to 2 T.

The voltage is used to accelerate the fibers to form a radial fiber arrangement in the inner ring. The magnetic field is used for deflection to create a circumferential fiber arrangement. Within the above-mentioned range of voltage and magnetic fields, the radial and circumferential forces can be covered to the maximum extent.

To further optimize the performance of the prefabricated film, optionally, the direction of the magnetic field lines of the magnetic field may be switchable and/or the receiving device may be rotatable.

Optionally, the method further includes removing residual solvent from the prefabricated film before performing the compositing step.

Optionally, the method for removing residual solvent from the prefabricated film involves drying the prefabricated film at 60 to 80° C. for 24 h to 48 h under a vacuum of 0.1 mbar/nitrogen atmosphere.

Optionally, the prefabricated film has a thickness of 0.05 to 0.2 mm.

The prefabricated film obtained by electrospinning is then composited with the second polyurethane to obtain a leaflet material. As an optional scheme for the compositing step, the compositing step may be carried out by dipping, that is, the second polyurethane is configured into a solution, and the prefabricated film is dip-coated in the second polyurethane solution to achieve the compositing of the prefabricated film and the second polyurethane. For the solvent that dissolves the second polyurethane, it is optional to choose a solvent that is conducive to the dissolution of the second polyurethane while maintaining the integrity of the scaffold of the prefabricated film. For example, when the first polyurethane with a hard segment content of 35% to 55% is selected, DMF may be chosen as the solvent for dissolving the second polyurethane.

Optionally, as an implementation method of the dip-coating, the prefabricated film is immersed in the second polyurethane solution for 4 to 6 seconds and then taken out, which can be repeated.

There is no restriction on the concentration of the second polyurethane and the number of dip-coating cycles for the prefabricated film, as long as the desired thickness is achieved. Specifically, the polymer valve leaflet material may be observed under a magnifying glass to see if there are any exposed pores or bare prefabricated films. If so, dip-coating is performed again.

Optionally, the mass percentage concentration of second polyurethane in the second polyurethane solution is in a range of 20% to 30%; and the requirement can be met by dip-coating 1 to 3 times within this concentration range.

As a specific example of the second polyurethane solution configuration, the second polyurethane is added in DMF solvent under a nitrogen atmosphere at 60° C. to 90° C., stirred for 4 to 16 hours, and fully dissolved to obtain a second polyurethane solution with a mass percentage concentration of 20% to 30%.

Optionally, during each dipping process, the prefabricated film is taken out from the second polyurethane solution after dipping for 4 to 6 seconds, degassed at 40° C. to 60° C. under vacuum for 3 to 8 minutes, and dried upright in an inert gas atmosphere at 60° C. to 80° C. for 10 to 20 hours.

The present disclosure further provides a polymer valve leaflet material prepared by the aforementioned method. The polymer valve leaflet material of the present disclosure may be used for interventional heart valve prostheses, such as those implanted through minimally invasive intervention, and may also be used for surgical heart valve prostheses, such as those implanted through surgical implantation.

The present disclosure further provides a valve prosthesis, including a stent and a leaflet material sewn on the stent, wherein the leaflet material is prepared by the method.

Optionally, the valve prosthesis is a heart valve prosthesis.

This disclosure uses an electrospinning valve as a molding substrate, which solves the problem of uneven leaflet thickness caused by the traditional impregnation process. At the same time, both impregnation and electrospinning are 3D stereoscopic molding methods, resulting in lower internal stress within the leaflet, which slows down the occurrence of calcification. That is, the leaflet obtained by the method of this disclosure can achieve a three-dimensional structure, ensure uniform leaflet thickness, and improve durability and fluid mechanics. Compared with the prior art, the leaflet material of this disclosure has at least one of the following beneficial effects:
(1) Good creep resistance;
(2) Strong edge tear resistance;
(3) Low stress and fatigue resistance during the cardiac cycle;
(4) Extended service life of the valve; and
(5) large EOA area and small regurgitation.

### BRIEF DESCRIPTION OF DRAWINGS

FIG. 1 is a schematic view of a trileaflet valve in a closed state.

FIG. 2 is a schematic view of a trileaflet valve in an open state.

FIG. 3 is a schematic view of a three-dimensional structure of a receiving device used in Example 1 and Example 2.

FIG. 4 is a schematic view of the three-dimensional structure of the receiving device used in Example 1 and Example 2 viewed at another angle.

FIG. 5 is a top view of the receiving device used in Example 1 and Example 2.

FIG. 6 is a schematic view of the electrospinning process in Example 2.

FIG. 7 is a diagram showing the distribution of electric and magnetic fields during the electrospinning process of Example 2.

FIG. 8 is a top view schematic of the fiber distribution during the electrospinning process in Example 2.

FIG. 9 is a schematic view of the appearance of a receiving device during the electrospinning process in Example 3.

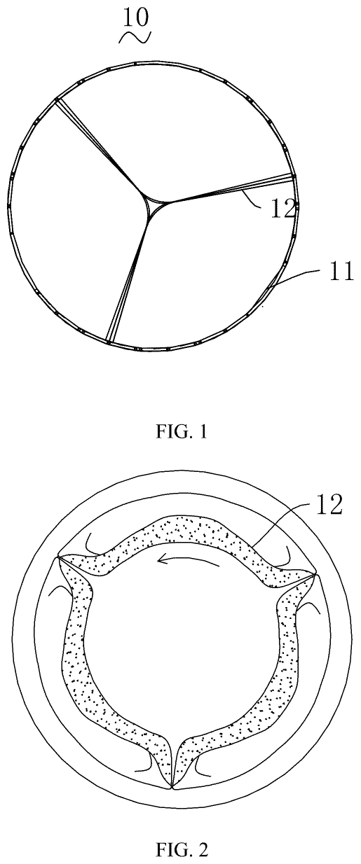

The reference signs shown in the figures are as follows:
10. valve leaflet; 11. fixed edge; 12. free edge;
20. first receiving device; 21. head part; 211. inclined wall; 22. tail part;
30. electrospinning needle;
40. electrode;
50. magnetic field;
60. second receiving device.

### DESCRIPTION OF EMBODIMENTS

Unless otherwise defined, all technical and scientific terms used herein have the same meaning as commonly understood by a person skilled in the art. The terms in the description of the present disclosure are used to describe specific embodiments, and not to limit the present disclosure.

### Example 1

(1) Preparation of the first polyurethane solution (by mass percentage): 45% polytetrahydrofuran (PTMO, molecular weight 1000), 44.91% isocyanate (MDI), 6.05% 1,4-butanediol (BDO) and 4.04% ethylenediamine (EDA) were added in a solvent (V_{THF}/V_{DMF}=3:1) and stirred for 8 h to prepare a homogeneous solution, which was a 10 wt % first polyurethane solution. That is, the first polyurethane content in the first polyurethane solution was 10 wt %. Among them, butanediol and ethylenediamine served as chain extenders, polytetrahydrofuran as a soft segment, and isocyanate along with chain extender as hard segments, with the content of hard segments of 55 wt %.

(2) The first polyurethane solution obtained in step (1) was electrospun onto a receiving device to obtain a prefabricated film with a thickness of 0.1 mm, which was then dried in a vacuum oven at 80° C. and 0.1 mbar for 48 h to remove residual solvent.

Electrospinning conditions were as follows: a humidity of 50%, a voltage of 15 kV, a propulsion speed of 1 mL/h, with a spinning distance of 20 cm and a needle gauge of 10 G.

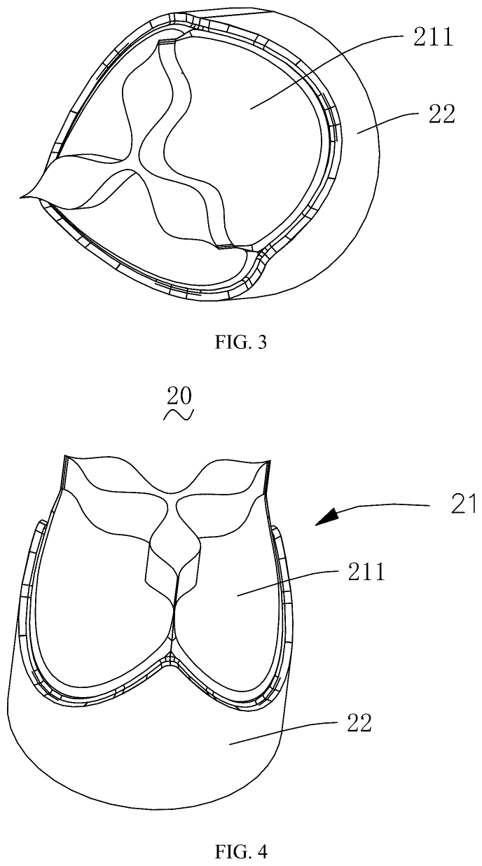

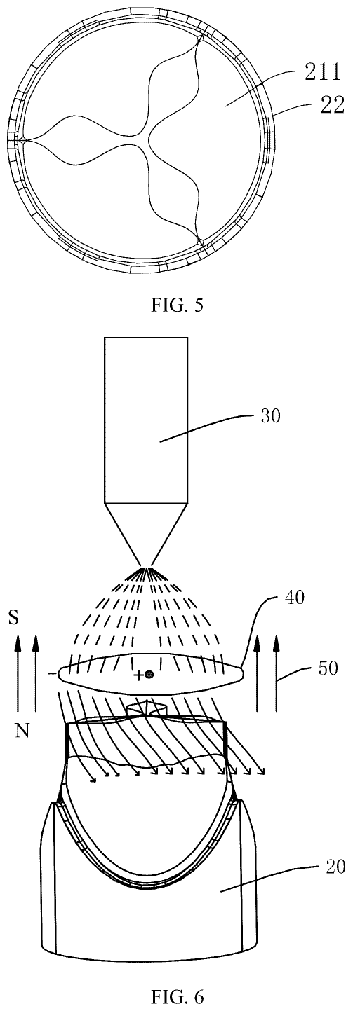

In this example, a first receiving device 20 used is a semi-open metal leaflet mold receiving device, with the structure schematically shown in FIGS. 3 to 5, which includes a head part 21 and a tail part 22 formed as a single piece. The head part has inclined walls 211, and the tail part is a rotating body. Specifically in this example, the tail part has a cylindrical structure, and the shape of each inclined wall is adapted to the transition state shape of the corresponding leaflet. In this example, the shape of the inclined wall corresponded to the leaflet being in a state where the opening area is ½ of the full opening. The end face of the head is planar.

(3) Preparation of second polyurethane solution (by mass percentage): 12% polydimethylsiloxane (PDMS, molecular weight 1000), 48% polytetrahydrofuran (PTMO, molecular weight 1000), 33.38% isocyanate (MDI) and 6.62% 1,2-butanediol (BDO) were dissolved in a solvent (DMAC) at 80° C. under a nitrogen atmosphere, stirred for 4 hours, and fully dissolved into a 20 wt % DMAc solution, which served as the second polyurethane solution. That is, the content of the second polyurethane in the second polyurethane solution was 20 wt %. The content of the hard segment (isocyanate and chain extender 1,2-butanediol) was 40 wt %.

(4) The prefabricated film obtained in step (2) was immersed in the second polyurethane (mass fraction: 20%) solution obtained in step (3), taken out from the solution after immersion for 5 seconds, degassed at 60° C. for 5 minutes under vacuum, and dried upright in an inert gas atmosphere at 80° C. for 20 hours. When observed under a magnifying glass, there were pores remaining not covered by the second polyurethane. The material was then immersed again in the second polyurethane (mass fraction of 20%) solution, taken out from the solution after immersion for 5 seconds, degassed at 60° C. for 5 minutes under vacuum, and dried upside down in an inert gas atmosphere at 80° C. for 20 hours. The thickness of the obtained polymer valve leaflet material was 0.15 mm.

### Example 2

(1) Preparation of the first polyurethane solution: same as in Example 1.

(2) The first polyurethane solution obtained in step (1) was electrospun onto a receiving device to obtain a prefabricated film on the receiving device with a thickness of 0.1 mm, which was then dried in a vacuum oven at 80° C. and 0.1 mbar for 48 h to remove residual solvent from the prefabricated film.

Electrospinning conditions were as follows: a humidity of 50%, a voltage of 15 kV, a propulsion speed of 1 mL/h, with a spinning distance of 20 cm, and a needle gauge of 10 G.

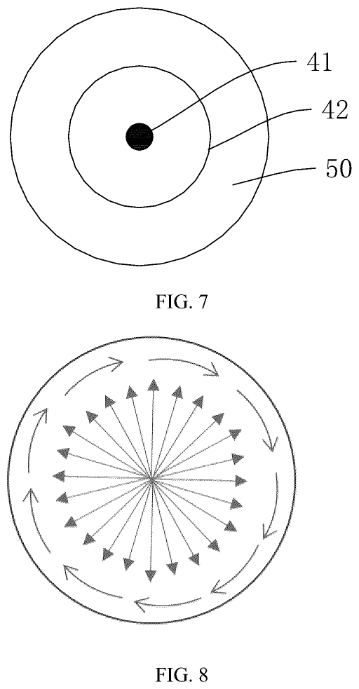

In this example, the receiving device is the same as in Example 1, and an electrode 40 is arranged between the first receiving device 20 and the electrospinning needle 30, providing a ring-shaped electric field for the spinning process. A ring-shaped magnetic field 50 is provided outside the ring-shaped electric field (there was no magnetic field in the entire vertical space within the electric field range), with the distribution diagram shown in FIG. 6. The electrode 40 includes a ring-shaped negative electrode and a positive electrode located at the center of the negative electrode, the directions of the magnetic field lines of the magnetic field 50 are directed from the receiving device to the spinning solution nozzle, the positive electrode of the electrode 40 is coaxial with the axis of the receiving device, and the plane where the negative electrode of the electrode 40 is located was perpendicular to the axis of the first receiving device 20, with the centers of the electric field, magnetic field, and receiving device on the same central axis, as shown in the top view schematic in FIG. 7.

In this example, the receiving device is circular in the top view with a radius of 26 mm. An electric field is added at a position 2 cm directly in front of the receiving device, the diameter (inner diameter) of the annular negative electrode is 24 mm, the positive electrode at the center is a solid electrode, the diameter of the positive electrode is 2 mm, and the thickness of the positive electrode and the negative electrode in the axial direction are both 1 mm.

The negative electrode is connected to the negative pole of the power supply, and the positive electrode is connected to the positive pole of the power supply. In this example, the voltage difference between the positive and negative poles is 20 kV, and the annular magnetic field is 0.5 T in magnitude. During the spinning process, the receiving device 20 rotates in a constant direction with a rotation speed of 2 r/min. When the fiber filament passes through the annular electric field, the electric field provides the fiber filament with a radially divergent force, and at the same time, the annular magnetic field provides the fiber filament with a circumferentially twisting force. The fiber distribution is finally obtained at the receiving device with the top view as shown in FIG. 8.

(3) Preparation of the second polyurethane solution: same as in Example 1.

(4) The obtained prefabricated film was immersed in a second polyurethane (mass fraction: 20%) solution, taken out from the solution after immersion for 5 seconds, degassed at 60° C. for 5 minutes under vacuum, and dried upright in an inert gas atmosphere at 80° C. for 20 hours. When observed under a magnifying glass, it was found that there were still pores not covered by the second polyurethane. The material was then immersed in a second polyurethane (mass fraction of 20%) solution, taken out from the solution after immersion for 5 seconds, degassed at 60° C. for 5 minutes under vacuum, and dried upside down in an inert gas atmosphere at 80° C. for 20 hours. The thickness of the obtained polymer valve leaflet material was 0.15 mm.

### Example 3

(1) Preparation of the first polyurethane solution: same as in Example 1.

(2) The first polyurethane solution obtained in step (1) was electrospun onto a second receiving device to obtain a prefabricated film on the receiving device with a thickness of 0.1 mm, which was then dried in a vacuum oven at 80° C. and 0.1 mbar for 48 h to remove residual solvent from the prefabricated film.

Electrospinning conditions were as follows: a humidity of 50%, a voltage of 15 kV, a propulsion speed of 1 mL/h, with a spinning distance of 20 cm, and a needle gauge of 10 G.

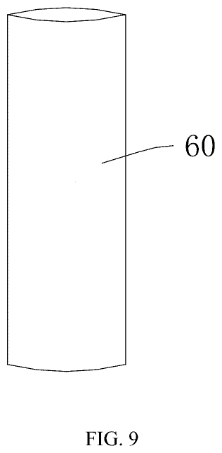

As shown in FIG. 9, the second receiving device 60 used in this example has a cylindrical structure, with a rotation speed of 2000 r/min.

(3) After detaching, one axial end of the cylindrical prefabricated film was bent inwardly to fit the surface of the mold (the shape of which is the same as the receiving device used in Example 1) to form the shape of leaflets. Then, the prefabricated film was heat-set by heating the mold (at a temperature of 180° C.).

(4) The contour of the prefabricated film was trimmed.

(5) Preparation of second polyurethane solution: same as in Example 1.

(6) The obtained prefabricated film was immersed in a second polyurethane (mass fraction: 20%) solution, and then taken out from the solution after immersion for 5 seconds, degassed at 60° C. for 5 minutes under vacuum, and dried upright in an inert gas atmosphere at 80° C. for 20 hours. Under a magnifying glass, it was observed that there were pores remaining not covered by the second polyurethane. The material was then immersed in a second polyurethane (mass fraction of 20%) solution, taken out from the solution after immersion for 5 seconds, degassed at 60° C. for 5 minutes under vacuum, and dried upside down in an inert gas atmosphere at 80° C. for 20 hours. The thickness of the obtained polymer valve leaflet material was 0.15 mm.

### Control Example 1

(1) Preparation of the second polyurethane solution: same as in Example 1.

(2) The second polyurethane solution was placed in a square container and dried at 80° C. for 24 h under a vacuum degree of 0.1 mbar to obtain a polyurethane membrane with a thickness of 0.15 mm.

### Example 4

(1) Preparation of the first polyurethane solution: same as in Example 1.

(2) The first polyurethane solution obtained in step (1) was electrospun onto a receiving device to obtain a prefabricated film on the receiving device with a thickness of 0.1 mm, which was dried in a vacuum oven at 80° C. and 0.1 mbar for 48 h to remove residual solvent.

Electrospinning conditions were as follows: a humidity of 50%, a voltage of 15 kV, a propulsion speed of 1 mL/h, a spinning distance of 20 cm, and a needle gauge of 10 G.

The receiving device in this example has a structure the same as that in Example 3, with a rotation speed of 50 r/min.

(3) Preparation of second polyurethane solution: same as in Example 1.

(4) After detaching, the cylindrical prefabricated film was bent inwardly and fixed onto a mold (the shape of which was the same as the receiving device used in Example 1), to form the shape of leaflets, and then the prefabricated film was heat-set by heating the mold.

(5) The contour of the prefabricated film was trimmed.

(6) Preparation of the second polyurethane solution: same as in Example 1.

(7) The obtained prefabricated film was immersed in the second polyurethane (mass fraction: 20%) solution, and then taken out from the solution after immersion for 5 seconds, degassed at 60° C. for 5 minutes under vacuum, and dried upright in an inert gas atmosphere at 80° C. for 20 hours. Under a magnifying glass, it was observed that there were still pores remaining not covered by the second polyurethane. The material was then immersed in a second polyurethane (mass fraction: 20%) solution, taken out from the solution after immersion for 5 seconds, degassed at 60° C. for 5 minutes under vacuum, and dried upside down in an inert gas atmosphere at 80° C. for 20 hours. The thickness of the obtained polymer valve leaflet material was 0.15 mm.

The fluid mechanics properties of Examples 1 to 4 and Control Example 1 were measured according to the relevant standard of ISO 5840. The specific test conditions and test results are shown in Table 1.

TABLE 1

| Leaflet inf. | | Test conditions | | | | | |
|---|---|---|---|---|---|---|---|
| | Leaflet | Heart | Cardiac | Mean | Pressure | Effective | Regurgitation |
| | size | rate | output | pressure | difference | opening | ratio |
| Leaflet No. | (mm) | (bpm) | (L/min) | (mmHg) | (mmHg) | area (cm² ) | (%) |
| Example 1 | | | | | | 1.81 | 14.32 |
| Example 2 | | | | | | 2.32 | 12.43 |
| Example 3 | 26 | 70 | 5 | 100 | 40 | 1.63 | 17.25 |
| Control | | | | | | 2.21 | 12.23 |
| Example 1 | | | | | | | |
| Example 4 | | | | | | 1.65 | 17.43 |

According to the results in Table 1, it can be seen that the effective opening areas of Examples 1 and 2 are larger than those of Examples 3 and 4, and the regurgitation ratios are smaller than those of Examples 3 and 4. This is because the receiving devices used in Examples 1 and 2 present the state when the leaflets are semi-open. In the semi-open state, the internal stress is zero, which can significantly reduce the maximum stress value of the valve leaflets during the cardiac cycle compared to the stress of zero when the flat valve is closed, thus greatly improving the fluid performance of the leaflets. In Example 2, the induced electrodes and induced magnetic fields are added during electrospinning to redistribute the fibers, so that the fibers are more similar to the natural leaflet morphology, further improving the fluid performance of the leaflets, and the fluid results are similar to those of unreinforced pure polyurethane materials.

The fatigue resistance of Examples 1 to 4 and Control Example 1 was measured according to ISO 5840 related standards, and the final test results are as follows:

Example 1: 800 million cycles, no tearing, with smaller pores at the seams.

Example 2: 700 million cycles, no tearing, with bigger pores at the seams.

Example 3: 600 million cycles, polyurethane peeling off the surface, with a small amount of tearing at the free edge.

Control Example 1: 8 million cycles, breakage, with tearing at the seam.

Example 4: 400 million cycles, polyurethane peeling off the surface, with larger pores at the seams.

It can be seen from the above results that the valves prepared in Examples 1 and 2 have good fatigue resistance. Comparing the results of Examples 3 and 4, it can be concluded that controlling the rotation speed of the receiving device is beneficial to improving the fatigue resistance of the leaflets.

The suture force and tear strength tests were carried out according to ASTM D412. The results are shown in Table 2:

TABLE 2

| Leaflet No. | Thickness (mm) | Suture force (N) | Tear strength (N/mm) |
|---|---|---|---|
| Example 1 | 0.15 | 4.5 | 145 |
| Example 2 | 0.15 | 8 | 123 |
| Example 3 | 0.15 | 6 | 110 |
| Control Example 1 | 0.15 | 2.5 | 50 |
| Example 4 | 0.15 | 4.5 | 141 |

In summary, as mentioned above, the leaflet prepared by the method of the present disclosure has significantly reduced maximum stress during one cardiac cycle due to three-dimensional molding method, which greatly extends the fatigue resistance of the leaflet. At the same time, the suture force of the leaflet at the suture is further increased by the induced electric field, the induced magnetic field, or the fiber anisotropy, making it difficult for the pores at the suture to further enlarge, thereby reducing the regurgitation amount of the leaflet after a period of use.

The above-described embodiments only illustrate several embodiments of the present disclosure, and the description thereof is specific and detailed, but should not be construed as limiting the scope of the patent disclosure. It should be noted that, for those of ordinary skill in the art, several modifications and improvements can be made without departing from the concept of the present disclosure, all of which fall into the protection scope of the present disclosure. Therefore, the protection scope of the present disclosure should be determined by the appended claims.

What is claimed is:

1. A preparation method for integrally forming a polymer valve leaflet material, comprising:
a spinning step, electrospinning a first polyurethane solution onto a receiving device to obtain a prefabricated film on the receiving device; and
a compositing step, compositing a second polyurethane with the prefabricated film to obtain the polymer valve leaflet material,
wherein the second polyurethane has a hard segment content 5% to 10% lower than the first polyurethane.

2. The preparation method for integrally forming a polymer valve leaflet material according to claim 1, wherein the first polyurethane is a polyurethane with a hard segment content of 35% to 55%, the first polyurethane is poorly soluble in a solvent system of the compositing step, and the second polyurethane is a biocompatible polyurethane.

3. The preparation method for integrally forming a polymer valve leaflet material according to claim 2, wherein the first polyurethane has a hard segment content of 40% to 55%, the hard segment is a polyurethane partially composed of diamines, and the second polyurethane is PDMS-PU.

4. The preparation method for integrally forming a polymer valve leaflet material according to claim 1, wherein the receiving device comprises a head part with inclined walls, and a shape of each inclined wall is adapted to a shape of corresponding leaflet in a transitional state.

5. The preparation method for integrally forming a polymer valve leaflet material according to claim 4, wherein each leaflet comprises a fixed edge and a free edge, and in the transition state, two adjacent leaflets are partially close to each other, and close portions comprise:
a portion of the free edge adjacent to the fixed edge of each leaflet; and
a portion of the free edge of each leaflet adjacent to a center of its own length.

6. The preparation method for integrally forming a polymer valve leaflet material according to claim 5, wherein when the leaflets are in a closed state, total length of two portions that are partially close to each other is approximately 0.1 to 0.4 times total length of the free edge, and the shape of the inclined wall corresponds to a state where an opening area of the leaflet is ¼ to ¾ of full opening.

7. The preparation method for integrally forming a polymer valve leaflet material according to claim 4, wherein the receiving device further comprises a tail part connected with the head part, the tail part is a rotating body as a whole, and an outer peripheral surface of the tail part is provided for electrostatic spinning to form skirts connected with the fixed edge of the leaflet.

8. The preparation method for integrally forming a polymer valve leaflet material according to claim 7, wherein the head and the tail parts are formed in one piece, and an end face of the head part is planar.

9. The preparation method for integrally forming a polymer valve leaflet material according to claim 4, wherein an electric field is applied through an electrode directly above the head part of the receiving device, and the electrode comprises a ring-shaped negative electrode and a positive electrode located at a center of the negative electrode; wherein a plane where the negative electrode is located is perpendicular to an axis of the receiving device, and the positive electrode is aligned with the axis of the receiving device.

10. The preparation method for integrally forming a polymer valve leaflet material according to claim 9, wherein a distance between the electrode and the head part of the receiving device is in a range of 1 to 3 cm along a height direction, an inner diameter of the negative electrode is in a range of 10 mm to 30 mm, and a diameter of the positive electrode is in a range of 2 mm to 5 mm.

11. The preparation method for integrally forming a polymer valve leaflet material according to claim 9, wherein during the electrospinning process, the receiving device is in a magnetic field environment, magnetic field lines are directed from the receiving device upward to the electrode, a magnitude of the magnetic field is in a range of 0.01 T to 2 T, or a voltage between the positive and negative electrodes is in a range of 15 to 30 kV.

12. The preparation method for integrally forming a polymer valve leaflet material according to claim 11, wherein directions of the magnetic field lines of the magnetic field are switchable and/or the receiving device is rotatable.

13. The preparation method for integrally forming a polymer valve leaflet material according to claim 1, wherein an outer periphery of the receiving device is a cylindrical surface, which rotates at a high speed during operation to form a cylindrical prefabricated film by electrostatic spinning; and
for the cylindrical prefabricated film, one axial end thereof is bent inwardly before compositing, so that a shape after bending corresponds to that of the leaflet being in a closed state and/or transition state, and a rotation speed of the receiving device is in a range of 1500 to 2500 r/min.

14. The preparation method for integrally forming a polymer valve leaflet material according to claim 1, wherein a mass percentage concentration of the first polyurethane in the first polyurethane solution is in a range of 8% to 18%, a solvent of the first polyurethane solution is a THF/DMF mixed solvent with a volume ratio of 1:1 to 5:1, and wherein the first polyurethane is stirred in the solvent for 4 to 16 hours to prepare a homogeneous solution.

15. The preparation method for integrally forming a polymer valve leaflet material according to claim 1, wherein during the electrospinning process, control conditions are set as follows: a humidity of 45% to 55%, a voltage of 10 kV to 25 kV, a propulsion speed of 0.5 mL/h to 1 mL/h, with a spinning distance of 15 cm to 25 cm and a needle gauge of 10 G to 30 G.

16. The preparation method for integrally forming a polymer valve leaflet material according to claim 1, further comprising removing a residual solvent from the prefabricated film before performing the compositing step, wherein the step for removing the residual solvent from the prefabricated film comprises drying the prefabricated film at 60° C. to 80° C. for 24 h to 48 h under a vacuum of 0.1 mbar/nitrogen atmosphere.

17. The preparation method for integrally forming a polymer valve leaflet material according to claim 1, wherein a thickness of the prefabricated film is in a range of 0.05 mm to 0.2 mm.

18. The preparation method for integrally forming a polymer valve leaflet material according to claim 1, wherein the compositing step adopts dip-coating comprising: configuring the second polyurethane into a solution, dip-coating the prefabricated film in the second polyurethane solution, and completing composite of the prefabricated film and the second polyurethane;
wherein during the dip-coating, the prefabricated film is immersed in the second polyurethane solution for 4 to 6 seconds and then taken out; and
wherein a mass percentage concentration of the second polyurethane in the second polyurethane solution is in a range of 20% to 30%, and the dip-coating is performed 1 to 3 times.

19. A polymer valve leaflet material prepared by the preparation method according to claim 1.

20. A heart valve prosthesis, comprising a stent and a valve material sewn on the stent, wherein the valve material is the polymer valve leaflet material according to claim 19.
